(19) United States
(12) Patent Application Publication
(10) Pub. No.: US 2007/0298086 A1
(43) Pub. Date: Dec. 27, 2007

(54) Tussi bag

(76) Inventor: Walter Ray Deal, Clearwater, FL (US)

Correspondence Address: Walter Ray Deal MD, 1262 Brookside Drive, Clearwater, FL, 33764, US

(21) Appl. No.: 11/474,869

(22) Filed: Jun. 26, 2006

(51) Int. Cl. A61K 9/70

(52) U.S. Cl. 424/443

(57) ABSTRACT

The Tussi Bag would provide a means whereby an infected individual would be able to reduce the probability of the spread of his/her respiratory infection. The Tussi Bag could be produced economically giving it unlimited availability. Individuals of almost any age would be able to successfully use this product. The design concept although simplistic in nature is still based upon sound medical principles:

(a) Capture the infectious respiratory droplets by directing the cough or sneeze into an impervious container.

(b) Expose these organisms to a chemical to which they are known to be sensitive.

(c) Devise an appropriate means of disposal of the contaminated materials once the infection has run its course.

### BACKGROUND OF THE INVENTION

1. Field of Invention

This application relates to a sanitary bag, particularly one used in the prevention of the spread of contagious respiratory infections.

2.Objects and Advantages

To design a new concept for protection against the spread of infections caused by coughing and sneezing by providing a reservoir into which one directs the sneeze or cough. Prior to this approach, the best advice offered by the medical field was:
(a) Cover your mouth with your hand when you cough. It was then determined that the infected hand spreads the infection as readily as simply coughing into the air.
(b) Cough into your sleeve. This method soon revealed that the infectious particles would be harbored within the clothes resulting in the spread of the infection by all contact with that individual.

### SUMMARY

It is becoming very obvious that the medical world is not very adept at stopping respiratory infections once they have started. Medical experts warn that these respiratory infections can become pandemic with devastating consequences. A new emphasis must be placed on preventative measures. The Tussi Bag offers the first step in this new approach.

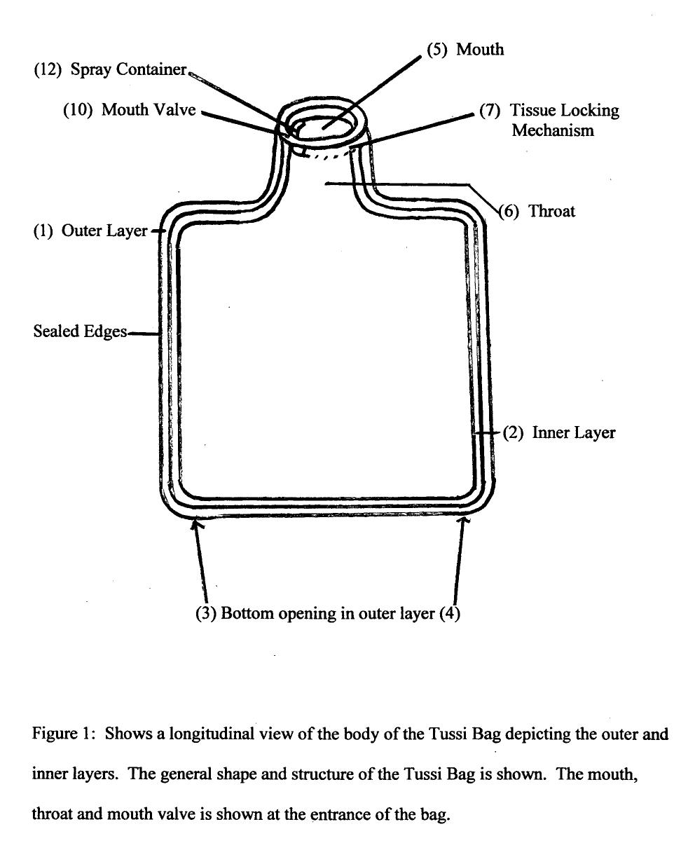

### DESCRIPTION OF FIGS. 1 THROUGH 7

FIG. 1: Shows a longitudinal view of the body of the Tussi Bag depicting the outer layer (1) and inner layer (2). The outer layer (1) is an impervious bag and will retain any infectious materials introduced through its mouth (5) and passing through the throat (6). The mouth (5) is the opening at the top that a user would open to cough or sneeze into to prevent the spread of the infectious materials contained within that cough or sneeze. The inner layer (2) is actually a tissue bag with an opening at the top that aligns with the opening at the top of the outer layer. The inner layer is tissue-like and chemically impregnated with an antiseptic and antibiotic substance to kill any infectious agent it contacts. The inner tissue layer is introduced into the Tussi Bag through the bottom by opening the outer layer (1) along the complete length between (3) and (4). The opening and closing of the outer layer in section 3 to 4 is by a zip lock-type mechanism The inner layer is disposed of after an appropriate duration of usage by removal through that same opening.

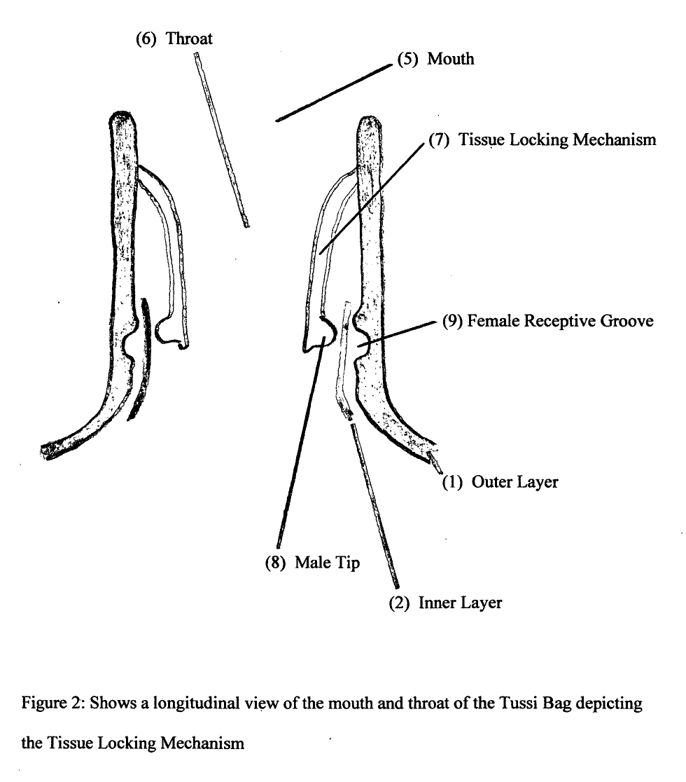

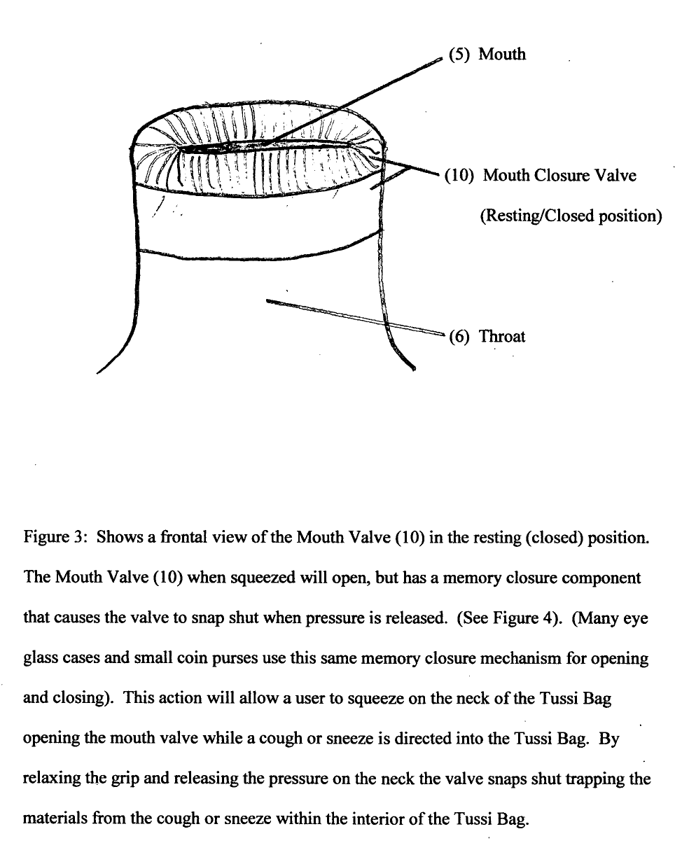

FIG. 2: Shows a longitudinal view of the mouth of the Tussi Bag (5) depicting the Tissue Locking Mechanism (7). By pressing the Male Tip (8) of the Tissue Locking Mechanism into the Female Receptive Groove (9) the inner tissue layer (2) is trapped and held in place. The tissue locking mechanism is attached to the inside of each side of the mouth of the outer layer and thereby opens the inner tissue bag whenever the mouth of the Tussi Bay is opened by the Mouth Valve. (See FIG. 3.)

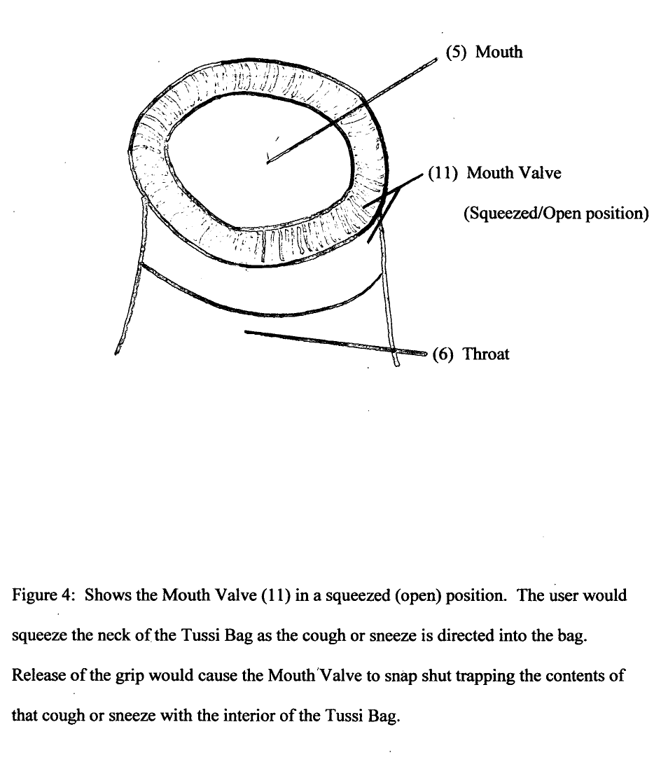

FIG. 3: Shows the Mouth Valve (10) in the resting (closed) position. The Mouth Valve when squeezed will open, but has a memory closure component that causes the valve to snap shut when pressure is released. (See FIG. 4). This action will allow a user to squeeze on the neck of the Tussi Bag opening the mouth valve while a cough or sneeze is directed into the Tussi Bag. By relaxing the grip and releasing the pressure on the neck the valve snaps shut trapping the materials from the cough or sneeze within the interior of the Tussi Bag.

FIG. 4: Shows the Mouth Valve (10) in a squeezed (open) position. The user would squeeze the neck of the Tussi Bag as the cough or sneeze is directed into the bag. Release of the grip would cause the Mouth Valve to snap shut trapping the contents of that cough or sneeze with the interior of the Tussi Bag.

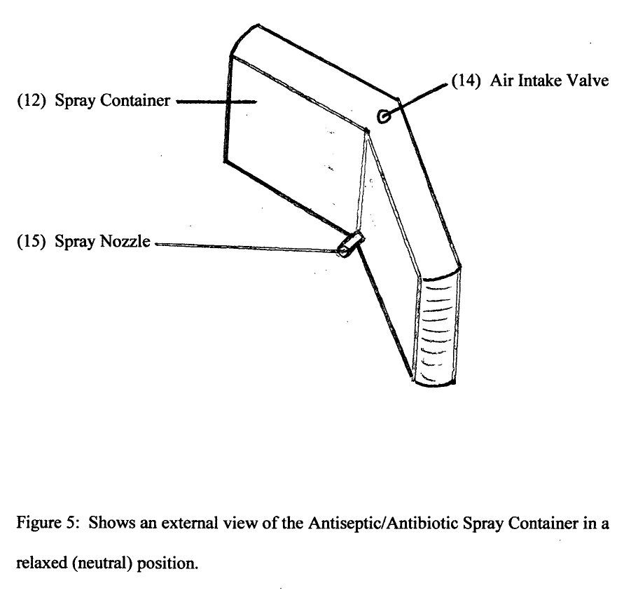

FIG. 5: Shows an exterior view of the Antiseptic/Antibiotic Spray Container (12) in a relaxed (neutral) position. The spray container (12) is attached to the bottom of the mouth valve and is activated by the movement of the mouth valve. When the mouth valve is in the position as shown in (11), the spray container will assume the shape depicted in FIG. 5. The assuming of this shape lessens the internal pressure within the spray container causing air to be drawn in through the air intake valve (14).

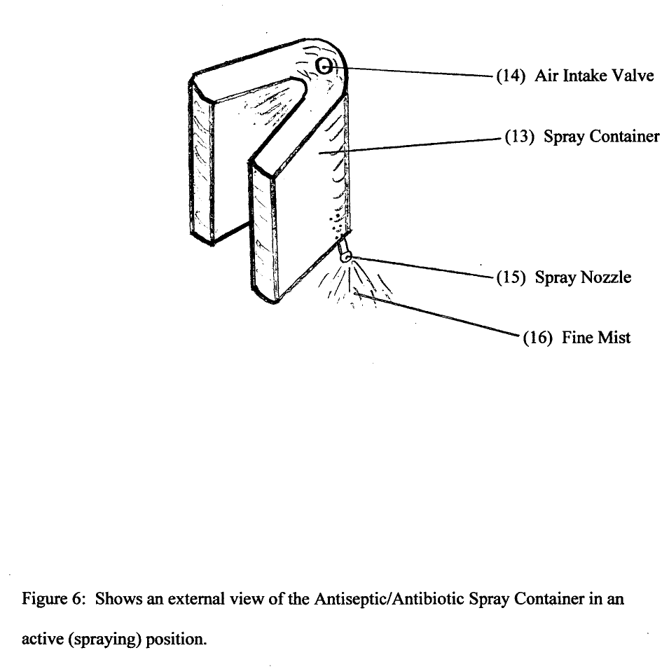

FIG. 6: Shows an exterior view of the Antiseptic/Antibiotic Stray Container (13) in an active (spraying) position. When the mouth valve is in the position as shown in (10), the spray container will assume the shape as depicted in FIG. 6. In this position due to the increase of internal pressure of the spray container, a fine mist (16) of Antiseptic/antibiotic chemical is sprayed through the Spray Nozzle (15) into the throat of the Tussi Bag.

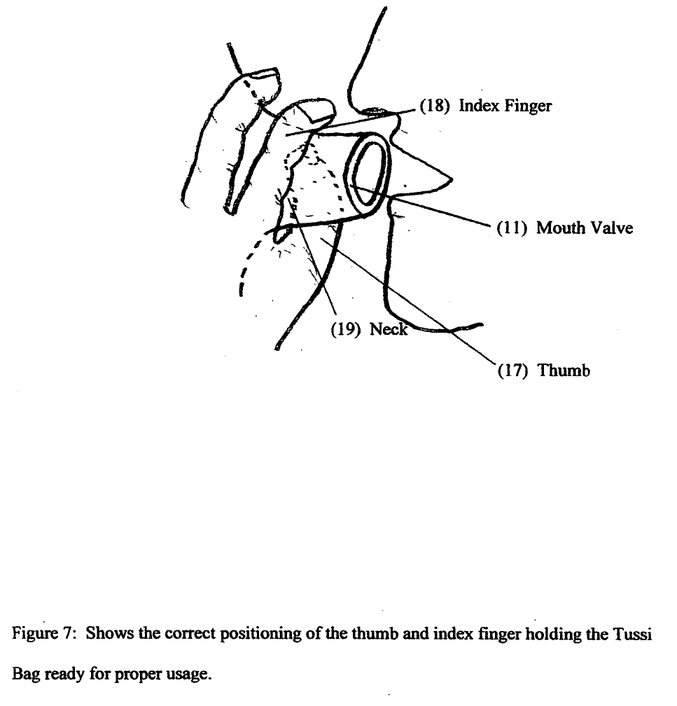

FIG. 7: Shows the correct positioning of the thumb (17) and index finger (18) grasping the neck (19) of the Tussi Bag with proper grip causing the Mouth valve (11) to open while the person coughs into the Tussi Bag. The Tussi Bag can be carried within an inside jacket pocket or purse and would be retrieved by grasping it in this position making it ready for immediate usage.

I. I claim this product is a new and original concept of a unique design of a: portable sanitary bag:
(a) having the potential of being carried in a pocket, a purse or any small container by an infected person, and
(b) having all edges sealed except for a mouth-like opening at the top and a means at the bottom to remove the contaminated tissue inner lining,
(c) having at the top a mouth valve that can be manually opened and closed and a means to adhere the inner tissue lining.
(d) allowing an infected person to raise the bag to his/her mouth to cough and sneeze into the bag, because
(e) squeezing gently on the neck of the bag opens the valve and relaxing that grip allows the valve to instantly close,
(f) causing a fine antiseptic and antibiotic spray to be released just beneath the valve area further enhancing the sterilization of the interior of the bag.

II. I claim this product is designed to capture, contain and kill the infectious organisms produced by coughing and sneezing:
(a) having an impervious outer layer preventing the escape of any infectious materials from the bag,
(b) having a tissue-like inner layer saturated with an antiseptic and antibiotic solution,
(c) killing the infectious organisms contained within the bag.

III. I claim this product has infection prevention potential:
(a) having an outer layer that can be sterilized and used repeatedly,
(b) having a chemically impregnated inner layer that can be removed and replaced,
(c) having an antiseptic and antibiotic spray container beneath the valve that can be replaced as needed,
(d) acknowledging that many infectious diseases are spread via infected respiratory droplets, and
(e) knowing that we have limited ability to stop many of the threatening contagious diseases once they have started,
(f) necessitating greater emphasis being placed on preventative medicine, and
(g) believing this product could be a significant advancement in the direction of preventative medicine for contagious respiratory infections.
